(19) United States
(12) Patent Application Publication
(10) Pub. No.: US 2025/0174330 A1
(43) Pub. Date: May 29, 2025

(54) CHEMICAL INFORMATION BASED ALERT STATE DETERMINATION

(71) Applicant: Cardiac Pacemakers, Inc., St. Paul, MN (US)

(72) Inventors: Pramodsingh Hirasingh Thakur, Woodbury, MN (US); Yingbo Li, Woodbury, MN (US); Michael J. Kane, St Paul, MN (US); Viktoria A. Averina, Shoreview, MN (US)

(21) Appl. No.: 18/958,347

(22) Filed: Nov. 25, 2024

Related U.S. Application Data
(60) Provisional application No. 63/604,054, filed on Nov. 29, 2023.

(51) Int. Cl. G16H 20/10; A61B 5/00; G16H 10/60; G16H 50/30

(52) U.S. Cl. CPC G16H 20/10; A61B 5/686; A61B 5/742; A61B 5/746; G16H 10/60; G16H 50/30

(57) ABSTRACT

Systems and methods are disclosed to determine an alert state for a health index using the health index and one or more pieces of chemical information. The chemical information can include potassium level, creatinine level, etc. The determined alert state can be provided to a user or a process.

### CLAIM OF PRIORITY

This application claims the benefit of U.S. Provisional Application No. 63/604,054, filed on Nov. 29, 2023, which is hereby incorporated by reference in its entirety.

### TECHNICAL FIELD

This document relates generally to medical devices and more particularly to determining or adjusting an alert state using a health index and chemical information.

### BACKGROUND

Ambulatory medical devices (AMDs), including implantable, subcutaneous, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs.

### SUMMARY

Systems and methods are disclosed to determine an alert state for a health index using the health index and one or more pieces of chemical information. The chemical information can include potassium level, creatinine level, etc. The determined alert state can be provided to a user or a process.

An example of subject matter (e.g., a medical device system) may comprise a signal receiver circuit configured to receive physiologic information of a patient and an assessment circuit configured to determine a health index for the patient as a function of the received physiologic information, wherein the signal receiver circuit is configured to receive chemical information of the patient, and wherein the assessment circuit is configured to determine an alert state of the patient using the determined health index and the received chemical information.

In an example, the health index includes a composite health index, and the assessment circuit is configured to determine the composite health index for the patient as a function of at least two features of the received physiologic information.

In an example, which may be combined with any one or more of the previous examples, to determine the alert state of the patient, the assessment circuit is configured to determine a first alert state of the patient using a value of the determined health index and a health index alert threshold, and determine a second alert state based on the determined first alert state and the received chemical information of the patient.

In an example, which may be combined with any one or more of the previous examples, the second alert state is one of a plurality of guideline-directed medical therapy (GDMT) alert states associated with a therapy adjustment, wherein the assessment circuit is configured to provide an output of the determined alert state to a user interface for display to a user or to a control circuit to control or adjust a process or function of the medical device system.

In an example, which may be combined with any one or more of the previous examples, the assessment circuit is configured to determine first and second chemical parameters using the received chemical information, wherein the received chemical information comprises at least one of potassium information or creatinine information of the patient, wherein the first and second chemical parameters comprise indications of relative high or low values of the received chemical information with respect to one or more thresholds, wherein to determine the first alert state, the assessment circuit is configured to determine that the value of the determined health index is above the health index alert threshold, and wherein the assessment circuit is configured to determine the second alert state based on the determined first alert state and at least one of the determined first and second chemical parameters.

In an example, which may be combined with any one or more of the previous examples, to determine the second alert state includes to determine that the potassium information of the patient is below a potassium threshold and the creatinine information of the patient is below a creatinine threshold, and wherein the assessment circuit is configured to provide a control signal to provide or increase a potassium sparing diuretic in response to determining the second alert state.

In an example, which may be combined with any one or more of the previous examples, to determine the second alert state includes to determine that the potassium information of the patient is above a potassium threshold and the creatinine information of the patient is below a creatinine threshold, and wherein the assessment circuit is configured to provide a control signal to provide or increase a thiazide diuretic in response to determining the second alert state.

In an example, which may be combined with any one or more of the previous examples to determine the second alert state includes to determine that the creatinine information of the patient is above a creatinine threshold, and wherein the assessment circuit is configured to provide a control signal to provide or increase a vasodilator in response to determining the second alert state.

In an example, which may be combined with any one or more of the previous examples, the health index includes a heart failure index and the health index alert threshold includes a heart failure alert threshold.

In an example, which may be combined with any one or more of the previous examples, a method can include receiving, using a signal receiver circuit, physiologic information of a patient, determining, using an assessment circuit, a health index for the patient as a function of the received physiologic information, receiving, using the signal receiver circuit, chemical information of a patient, and determining, using the assessment circuit, an alert state of the patient using the determined health index and the received chemical information.

In an example, which may be combined with any one or more of the previous examples, determining the alert state of the patient includes determining a first alert state of the patient using a value of the determined health index and a health index alert threshold, and determining a second alert state based on the determined first alert state and the received chemical information of the patient.

In an example, which may be combined with any one or more of the previous examples, a method can include providing, using the assessment circuit, an output of the determined alert state to a user interface for display to a user or to a control circuit to control or adjust a process or function of a medical device system, where the second alert state is one of a plurality of guideline-directed medical therapy (GDMT) alert states associated with a therapy adjustment.

In an example, which may be combined with any one or more of the previous examples, a method can include determining, using the assessment circuit, first and second chemical parameters using the received chemical information, where the received chemical information comprises at least one of potassium information or creatinine information of the patient, where the first and second chemical parameters comprise indications of relative high or low values of the received chemical information with respect to one or more thresholds, where determining the first alert state includes determining that the value of the determined health index is above the health index alert threshold, and where determining the second alert state includes determining the second alert state based on the determined first alert state and at least one of the determined first and second chemical parameters.

In an example, which may be combined with any one or more of the previous examples, determining the second alert state includes determining that the potassium information of the patient is below a potassium threshold and the creatinine information of the patient is below a creatinine threshold, and providing, in response to determining the second alert state, a control signal to provide or increase a potassium sparing diuretic.

In an example, which may be combined with any one or more of the previous examples, a method can include determining the second alert state includes determining that the potassium information of the patient is above a potassium threshold and the creatinine information of the patient is below a creatinine threshold, and providing, in response to determining the second alert state, a control signal to provide or increase a thiazide diuretic.

In an example, which may be combined with any one or more of the previous examples, a medical devices system may comprise a signal receiver circuit configured to receive physiologic information of a patient and an assessment circuit configured to determine a health index for the patient as a function of the received physiologic information, wherein the signal receiver circuit is configured to receive chemical information of the patient, and wherein the assessment circuit is configured to determine an alert state of the patient using the determined health index and the received chemical information.

In an example, the health index includes a composite health index, and the assessment circuit is configured to determine the composite health index for the patient as a function of at least two features of the received physiologic information.

In an example, which may be combined with any one or more of the previous examples, to determine the alert state of the patient, the assessment circuit is configured to determine a first alert state of the patient using a value of the determined health index and a health index alert threshold, and determine a second alert state based on the determined first alert state and the received chemical information of the patient.

In an example, which may be combined with any one or more of the previous examples, the second alert state is one of a plurality of guideline-directed medical therapy (GDMT) alert states associated with a therapy adjustment, wherein the assessment circuit is configured to provide an output of the determined alert state to a user interface for display to a user or to a control circuit to control or adjust a process or function of the medical device system.

In an example, which may be combined with any one or more of the previous examples, the assessment circuit is configured to determine first and second chemical parameters using the received chemical information, wherein the received chemical information comprises at least one of potassium information or creatinine information of the patient, wherein the first and second chemical parameters comprise indications of relative high or low values of the received chemical information with respect to one or more thresholds, wherein to determine the first alert state, the assessment circuit is configured to determine that the value of the determined health index is above the health index alert threshold, and wherein the assessment circuit is configured to determine the second alert state based on the determined first alert state and at least one of the determined first and second chemical parameters.

In an example, which may be combined with any one or more of the previous examples, to determine the second alert state includes to determine that the potassium information of the patient is below a potassium threshold and the creatinine information of the patient is below a creatinine threshold, and wherein the assessment circuit is configured to provide a control signal to provide or increase a potassium sparing diuretic in response to determining the second alert state.

In an example, which may be combined with any one or more of the previous examples, to determine the second alert state includes to determine that the potassium information of the patient is above a potassium threshold and the creatinine information of the patient is below a creatinine threshold, and wherein the assessment circuit is configured to provide a control signal to provide or increase a thiazide diuretic in response to determining the second alert state.

In an example, which may be combined with any one or more of the previous examples to determine the second alert state includes to determine that the creatinine information of the patient is above a creatinine threshold, and wherein the assessment circuit is configured to provide a control signal to provide or increase a vasodilator in response to determining the second alert state.

In an example, which may be combined with any one or more of the previous examples, the health index includes a heart failure index and the health index alert threshold includes a heart failure alert threshold.

In an example, which may be combined with any one or more of the previous examples, a method includes receiving, using a signal receiver circuit, physiologic information of a patient, determining, using an assessment circuit, a health index for the patient as a function of the received physiologic information, receiving, using the signal receiver circuit, chemical information of a patient, and determining, using the assessment circuit, an alert state of the patient using the determined health index and the received chemical information.

In an example, which may be combined with any one or more of the previous examples, the health index includes a composite health index, and determining the health index includes determining the composite health index for the patient as a function of at least two features of the received physiologic information.

In an example, which may be combined with any one or more of the previous examples, determining the alert state of the patient includes determining a first alert state of the patient using a value of the determined health index and a health index alert threshold, and determining a second alert state based on the determined first alert state and the received chemical information of the patient.

In an example, which may be combined with any one or more of the previous examples, the method includes providing, using the assessment circuit, an output of the determined alert state to a user interface for display to a user or to a control circuit to control or adjust a process or function of a medical device system, wherein the second alert state is one of a plurality of guideline-directed medical therapy (GDMT) alert states associated with a therapy adjustment.

In an example, which may be combined with any one or more of the previous examples, the method includes determining, using the assessment circuit, first and second chemical parameters using the received chemical information, wherein the received chemical information comprises at least one of potassium information or creatinine information of the patient, wherein the first and second chemical parameters comprise indications of relative high or low values of the received chemical information with respect to one or more thresholds, wherein determining the first alert state includes determining that the value of the determined health index is above the health index alert threshold, and wherein determining the second alert state includes determining the second alert state based on the determined first alert state and at least one of the determined first and second chemical parameters.

In an example, which may be combined with any one or more of the previous examples, determining the second alert state includes determining that the potassium information of the patient is below a potassium threshold and the creatinine information of the patient is below a creatinine threshold, and providing, in response to determining the second alert state, a control signal to provide or increase a potassium sparing diuretic.

In an example, which may be combined with any one or more of the previous examples, determining the second alert state includes determining that the potassium information of the patient is above a potassium threshold and the creatinine information of the patient is below a creatinine threshold, and providing, in response to determining the second alert state, a control signal to provide or increase a thiazide diuretic.

In an example, which may be combined with any one or more of the previous examples, determining the second alert state includes determining that the creatinine information of the patient is above a creatinine threshold, and providing, in response to determining the second alert state, a control signal to provide or increase a vasodilator.

In an example, which may be combined with any one or more of the previous examples, the health index includes a heart failure index and the health index alert threshold includes a heart failure alert threshold.

In an example, a system or apparatus may optionally combine any portion or combination of any portion of any one or more of the examples above to comprise “means for” performing any portion of any one or more of the functions or methods of the examples above, or at least one “non-transitory machine-readable medium” including instructions that, when performed by a machine, cause the machine to perform any portion of any one or more of the functions or methods of the examples above.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example medical device system.

FIG. 2 illustrates an example patient management system.

FIG. 3 illustrates an example method for using chemical information to determine an alert for a health index.

FIG. 4 illustrates an example method for using chemical information to determine a second alert for a health index.

FIG. 5 illustrates an example implantable medical device (IMD) electrically coupled to a heart.

FIG. 6 illustrates a block diagram of an example machine upon which any one or more of the techniques discussed herein may perform.

### DETAILED DESCRIPTION

Ambulatory medical devices can include or be configured to receive physiologic information from one or more sensors located within, on, or proximate to a body of a patient. Physiologic information of the patient can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient.

One or more health indexes can be determined, in certain examples, as a function of different physiologic information of the patient or various combinations thereof. Health indexes can include single-feature health indexes determined using a single feature or measure of a single type of physiologic information, or separately a composite health index determined using a combination of physiologic information, such as two or more separate features of different physiologic measures. For example, although respiratory rate and tidal volume are both respiratory information, they are separate features of respiratory information, such that a composite health index can be determined using respiratory rate and tidal volume. In contrast, a single-feature health index can be determined using respiratory information, such as using a trend or measure of tidal volume alone.

In certain examples, a health index can be a device-based index, such as determined using physiologic information detected from the patient without input of clinical information about the patient separate from that detected or sensed from the device, such as clinician diagnosis or determination of risk, patient history, patient age, comorbidities, prior hospitalization, type of implanted device, etc. In other examples, the health index can be a combination of a device-based and clinical-based mortality risk index, including or taking into account clinical information about the patient, such as clinician diagnosis or determination of risk, patient history, patient age, comorbidities, prior hospitalization, type of implanted device, etc. In an example, separate determinations can be made for different combinations of clinical information.

One example of a composite health index is a HeartLogic™ index, a HeartLogic™ in-alert time, or one or more other composite measurements or measures thereof. The HeartLogic™ index is a composite measurement of electrical and mechanical physiologic information of a patient from multiple ambulatory sensors, including S1 and S3 heart sounds, thoracic impedance, activity information, respiration information, and nighttime heart rate (nHR), and can be indicative of a heart failure status, a risk of heart failure event, or a worsening of the heart failure status or risk of heart failure event in the patient over time. The HeartLogic™ in-alert time is a measure of time that the HeartLogic™ index is above an alert threshold.

In certain examples, the HeartLogic™ index can be determined using different combinations or weightings of electrical and mechanical physiologic information, including one or more of S1 and S3 heart sounds, thoracic impedance, activity information, rapid shallow breathing index (RSBI), respiratory rate, and nighttime heart rate (nHR). In certain examples, the different combinations or weightings of the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.).

For example, if the risk stratifier is low, or below a first threshold, the HeartLogic™ index can be determined using a first combination of physiologic information. If the risk stratifier is high, or above a second threshold, the HeartLogic™ index can be determined using the first combination of physiologic information and a second combination of physiologic information, including additional information than included in the first combination. If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

Implantable and ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc.

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a ratio that measures the respiratory frequency divided by the relative tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical physiologic information, but different than cardiac electrical information. In other examples, respiration information of the patient can be determined using changes in activity or acceleration information and accordingly can be considered mechanical physiologic information.

Physiologic values or features, as described herein, can include one or more different measures of rate, amplitude, energy, etc., of different physiologic information over one or more time periods, such as representative daily values, etc. For example, heart sound values can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily values can be determined representative of an average daily value for the patient, either corresponding to a waking time or a 24-hour period, etc. Respiration values can include, among other things, a mean or median respiration rate, binned values of rates, and a representative value of specific rate bins, etc. Heart rate values can include an average nighttime heart rate, a minimum nighttime heart rate, etc.

The activity information can include an activity measurement of the patient, such as detected using an accelerometer, a posture sensor, a step counter, or one or more other activity sensors associated with an ambulatory medical device. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical physiologic information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

The present inventors have recognized, among other things, systems and methods to adjust or determine a health index alert state of the patient using chemical information, such as to increase a sensitivity or specificity of health index alert state determination, reduce false positive alert state determinations, alert state transitions or adjustments, or otherwise reduce storage or transmission of physiologic information associated or transitions associated with false positive alert state determinations, and power and processing resources associated with the same. In an example, a system can determine one or more pieces of chemical information, and use the chemical information to determine, adjust, or include additional information about the alert state or a determined condition of the patient.

In certain examples, the system may have an alert state (e.g., an in-alert state, an out-of-alert state, a priority alert state, etc.). The alert state may be provided to the patient, a clinician, or one or more other users or devices associated with the patient. The alert state may be determined using the health index, such as the HeartLogic™ index, and one or more features of the received chemical information. In an example, the health index can be determined using one or more features based on the received chemical information, and an alert state determined based on the health index may therefore be an alert state based on the health index and the received chemical information. In an example, the health index may not include any features based on the received chemical information, but instead the physiologic information not including received chemical information, and an alert state based on the health index may therefore not be an alert state based on the health index and the received chemical information.

In an example, the health index may have a value (e.g., a numerical value, etc.) and the value may be compared to one or more thresholds to determine the alert state of the system. The assessment circuit may be configured to compare the health index to one or more health index alert thresholds (e.g., a single health index alert threshold, first and second health index alert thresholds, etc.) to determine a first alert state of the patient. In an example, the health index may have a numerical value with higher values representing a worse health state of the patient and lower values representing a better health state of the patient. The health index alert threshold may represent a high threshold that results in an in-alert state being triggered if the value of the health index exceeds the health index alert threshold.

The health index alert threshold may be a fixed value, or it may be an adaptable threshold that varies based on one or more factors. In an example, the health index alert threshold may be fixed, but a value of the health index may be based in part on one or more relative factors (e.g., based on measurements from the patient over the past 30 days as opposed to being based on fixed values), which may result in the in-alert state threshold condition of the patient being relative even though the health index alert threshold is fixed.

Comparing the health index value to the health index alert threshold may determine a first alert state of the system. This first alert state may be based at least in part on the received chemical information if the health index includes or is determined using one or more features generated using the chemical information, or the first alert state may not be based on the received chemical information if the health index does not include or is not determined using any features generated using the chemical information. The system may generate a second alert state based on the determined first alert state and one or more features of the received chemical information. For example, the system may determine the second alert state by adjusting the determined first alert state (e.g., adjusting an in-alert state to an out-of-alert state, adjusting an in-alert state to a priority alert state, maintaining the first alert state, etc.), by augmenting the determined first alert state (e.g., appending one or more pieces of additional information to the alert state), etc. In an example, the system may determine the second alert state based on one or more of the determined first alert state, one or more features of the received chemical information, the health index (e.g., the health index value), one or more other pieces of received physiologic information, one or more features used in the health index, a historical value of one or more features, etc.

Augmenting the first alert state may include providing one or more additional pieces of information to a clinician. For example, the determined second alert state may provide the doctor with an indication to perform one or more actions (e.g., suggesting a drug or drug class from a plurality of options, suggesting an optimization of guideline-directed medical therapy (GDMT), etc.). The determined second alert state can be provided to a user interface for display to a user or to a control circuit to control or adjust a process or function of the system. In an example, the determined second alert state can include one or more of an indication or recommendation to administer or provide a class of drug or an indication or recommendation related to GDMT. A GDMT may provide a standard course of action to follow in the treatment of a patient. For, example, a GDMT may advise administration of a quantity of a drug or a rate of increase in dosage, etc. In an example, when received chemical information indicates that one or more of potassium levels are low or creatinine levels are low, the second alert state may include one or more of providing an indication to administer or provide a potassium sparing diuretic or providing an indication to deviate from GDMT (e.g., increase GDMT above a standard recommendation, increase a dose of a drug, adjust a rate of increase in a drug, etc.).

In an example, when received chemical information indicates that one or more of potassium levels are high or creatinine levels are low, such as with respect to first and second thresholds, in combination with a value of the determined health index indicating the first alert state, the second alert state may include one or more of providing an indication to administer or provide a thiazide diuretic or providing an indication to hold GDMT (e.g., hold GDMT at a standard recommendation, hold GDMT at a current level, etc.).

In an example, when received chemical information indicates that one or more of potassium levels are low or creatinine levels are high, the second alert state may include one or more of providing an indication to administer or provide a vasodilator (e.g., use a vasodilator as an alternative to a diuretic, use a vasodilator in addition to a diuretic, such as in combination with reducing the diuretic dosage, etc.). or providing an indication to hold or cut back GDMT (e.g., decrease GDMT below a standard recommendation, decrease a dose of a drug, decrease a rate of increase of a drug, etc.).

In an example, when received chemical information indicates that one or more of potassium levels are high or creatinine levels are high, the second alert state may include one or more of providing an indication to administer or provide a vasodilator or providing an indication to cut back GDMT.

In an example, when the received chemical information indicates that one or more of potassium or creatinine are at a normal level, the second alert state may not be given, or the second alert state may provide an indication to follow normal guidelines (e.g., provide diuretics and/or up-titrate GDMT).

In certain examples, the techniques described above or herein can be used in various combinations or permutations. For example, combinations or permutations of techniques described above or herein can be selected based upon patient history, clinician input, etc.

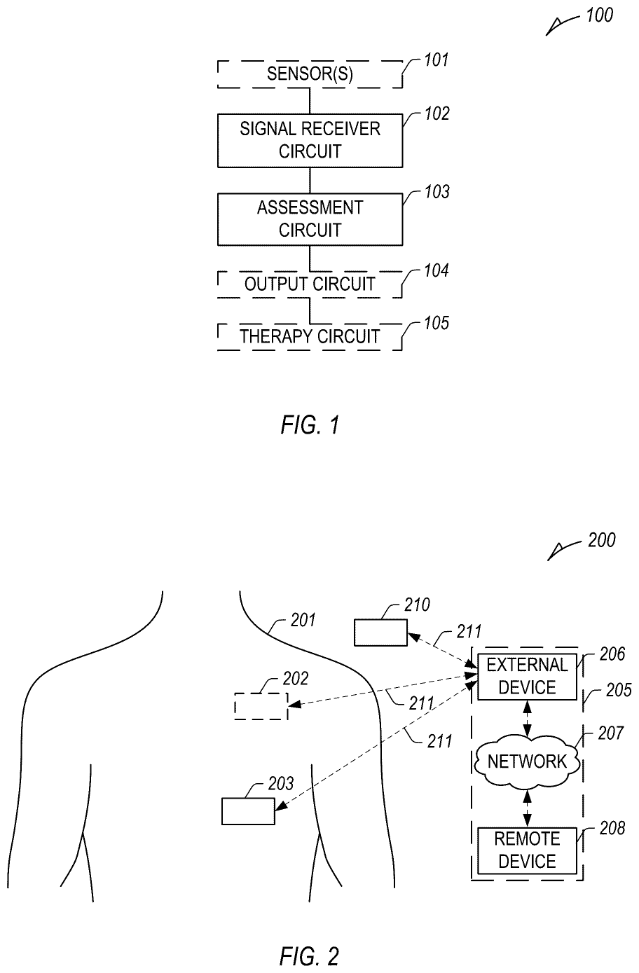

FIG. 1 illustrates an example system 100 (e.g., a medical device system). In an example, one or more aspects of the example system 100 can be a component of, or communicatively coupled to, a medical device, such as an implantable medical device (IMD), an insertable cardiac monitor, an ambulatory medical device (AMD), etc. The system 100 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 100 can include a single medical device or a plurality of medical devices implanted in a patient's body or otherwise positioned on or about the patient to monitor patient physiologic information of the patient using information from one or more sensors, such as a sensor 101. In an example, the sensor 101 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information; a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 100 can include a signal receiver circuit 102 and an assessment circuit 103. The signal receiver circuit 102 can be configured to receive physiologic information of a patient (or group of patients) from the sensor 101. The assessment circuit 103 can be configured to receive information from the signal receiver circuit 102, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 103 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 100 can include an output circuit 104 configured to provide an output to a user, or to cause an output to be provided to a user, such as through an output, a display, or one or more other user interface, the output including a score, a trend, an alert, or other indication. In other examples, the output circuit 104 can be configured to provide an output to another circuit, machine, or process, such as a therapy circuit 105 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 105 can include one or more of a stimulation control circuit, a cardiac stimulation circuit, a neural stimulation circuit, a dosage determination or control circuit, etc. In other examples, the therapy circuit 105 can be controlled by the assessment circuit 103, or one or more other circuits, etc. In certain examples, the assessment circuit 103 can include the output circuit 104 or can be configured to determine the output to be provided by the output circuit 104, while the output circuit 104 can provide the signals that cause the user interface to provide the output to the user based on the output determined by the assessment circuit 103.

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

For example, a change in modes can enable higher resolution sampling or an increase in the sampling frequency or number or types of sensors used to sense physiologic information leading up to and including a potential event. For example, different physiologic information is often sensed using non-overlapping time periods of the same sensor, in certain examples, at different sampling frequencies and power costs. In one example, heart sounds and patient activity can be detected using non-overlapping time periods of the same, single- or multi-axis accelerometer, at different sampling frequencies and power costs. In certain examples, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

FIG. 2 illustrates an example patient management system 200 and portions of an environment in which the patient management system 200 may operate. The patient management system 200 can perform a range of activities, including remote patient monitoring and diagnosis of a disease condition. Such activities can be performed proximal to a patient 201, such as in a patient home or office, through a centralized server, such as in a hospital, clinic, or physician office, or through a remote workstation, such as a secure wireless mobile computing device.

The patient management system 200 can include one or more medical devices, an external system 205, and a communication link 211 providing for communication between the one or more ambulatory medical devices and the external system 205. The one or more medical devices can include an ambulatory medical device (AMD), such as an implantable medical device (IMD) 202, a wearable medical device 203, or one or more other implantable, leadless, subcutaneous, external, wearable, or medical devices configured to monitor, sense, or detect information from, determine physiologic information about, or provide one or more therapies to treat various conditions of the patient 201, such as one or more cardiac or non-cardiac conditions (e.g., dehydration, sleep disordered breathing, etc.).

In an example, the implantable medical device 202 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 201. In another example, the implantable medical device 202 can include a monitor implanted, for example, subcutaneously in the chest of patient 201, the implantable medical device 202 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 202 can include an assessment circuit configured to detect or determine specific physiologic information of the patient 201, or to determine one or more conditions or provide information or an alert to a user, such as the patient 201 (e.g., a patient), a clinician, or one or more other caregivers or processes, such as described herein. The implantable medical device 202 can alternatively or additionally be configured as a therapeutic device configured to treat one or more medical conditions of the patient 201. The therapy can be delivered to the patient 201 via the lead system and associated electrodes or using one or more other delivery mechanisms. The therapy can include delivery of one or more drugs to the patient 201, such as using the implantable medical device 202 or one or more of the other ambulatory medical devices, etc. In some examples, therapy can include CRT for rectifying dyssynchrony and improving cardiac function in heart failure patients. In other examples, the implantable medical device 202 can include a drug delivery system, such as a drug infusion pump to deliver drugs to the patient for managing arrhythmias or complications from arrhythmias, hypertension, hypotension, or one or more other physiologic conditions. In other examples, the implantable medical device 202 can include one or more electrodes configured to stimulate the nervous system of the patient or to provide stimulation to the muscles of the patient airway, etc.

The wearable medical device 203 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 205 can include a dedicated hardware/software system, such as a programmer, a remote server-based patient management system, or alternatively a system defined predominantly by software running on a standard personal computer. The external system 205 can manage the patient 201 through the implantable medical device 202 or one or more other ambulatory medical devices connected to the external system 205 via a communication link 211. In other examples, the implantable medical device 202 can be connected to the wearable medical device 203, or the wearable medical device 203 can be connected to the external system 205, via the communication link 211. This can include, for example, programming the implantable medical device 202 to perform one or more of acquiring physiologic data, performing at least one self-diagnostic test (such as for a device operational status), analyzing the physiologic data, or optionally delivering or adjusting a therapy for the patient 201. Additionally, the external system 205 can send information to, or receive information from, the implantable medical device 202 or the wearable medical device 203 via the communication link 211. Examples of the information can include real-time or stored physiologic data from the patient 201, diagnostic data, such as detection of patient hydration status, hospitalizations, responses to therapies delivered to the patient 201, or device operational status of the implantable medical device 202 or the wearable medical device 203 (e.g., battery status, lead impedance, etc.). The communication link 211 can be an inductive telemetry link, a capacitive telemetry link, or a radio-frequency (RF) telemetry link, or wireless telemetry based on, for example, “strong” Bluetooth or IEEE 502.11 wireless fidelity “Wi-Fi” interfacing standards. Other configurations and combinations of patient data source interfacing are possible.

The external system 205 can include an external device 206 in proximity of the one or more ambulatory medical devices, and a remote device 208 in a location relatively distant from the one or more ambulatory medical devices, in communication with the external device 206 via a communication network 207. Examples of the external device 206 can include a medical device programmer. The remote device 208 can be configured to evaluate collected patient or patient information and provide alert notifications, among other possible functions. In an example, the remote device 208 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 208 can receive data from multiple patients. The data can be collected by the one or more ambulatory medical devices, among other data acquisition sensors or devices associated with the patient 201. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 208 may additionally include one or more locally configured clients or remote clients securely connected over the communication network 207 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 208, including the server and the interconnected clients, may also execute a follow-up scheme by sending follow-up requests to the one or more ambulatory medical devices, or by sending a message or other communication to the patient 201 (e.g., the patient), clinician or authorized third party as a compliance notification.

The communication network 207 can provide wired or wireless interconnectivity. In an example, the communication network 207 can be based on the Transmission Control Protocol/Internet Protocol (TCP/IP) network communication specification, although other types or combinations of networking implementations are possible. Similarly, other network topologies and arrangements are possible.

One or more of the external device 206 or the remote device 208 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 206 or the remote device 208 can include a respective display unit for displaying the physiologic or functional signals, or alerts, alarms, emergency calls, or other forms of warnings to signal the detection of arrhythmias. In some examples, the external system 205 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 205 can be implemented using hardware, software, firmware, or combinations thereof. Portions of the one or more ambulatory medical devices or the external system 205 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 210 can be configured to send information to or receive information from one or more of the ambulatory medical devices or the external system 205 using the communication link 211. In an example, the one or more ambulatory medical devices, the external device 206, or the remote device 208 can be configured to control one or more parameters of the therapy device 210. The external system 205 can allow for programming the one or more ambulatory medical devices and can receive information about one or more signals acquired by the one or more ambulatory medical devices, such as can be received via a communication link 211. The external system 205 can include a local external implantable medical device programmer. The external system 205 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected atrial fibrillation event, or combinations thereof.

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

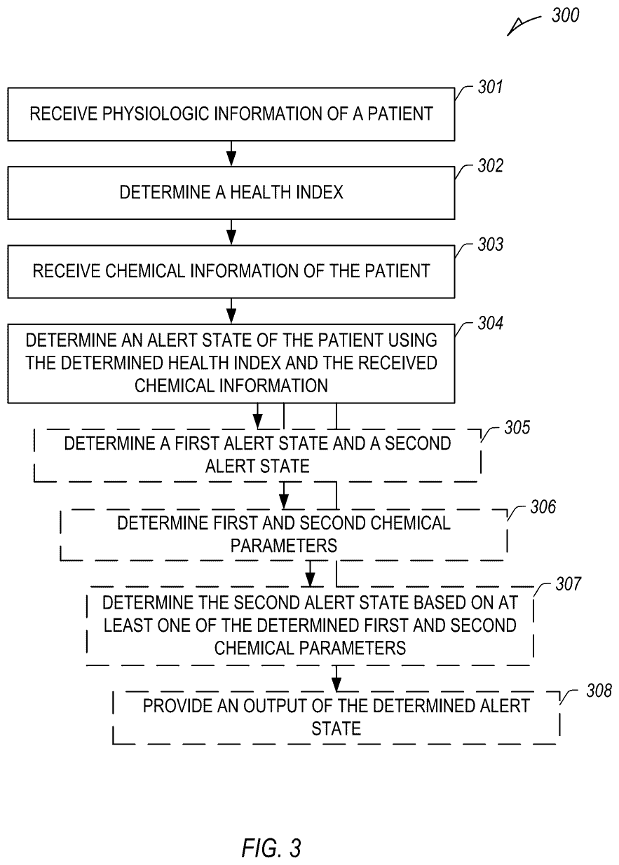

FIG. 3 illustrates an example method 300 for using chemical information in an alert for a health index, such as described herein.

At step 301, physiologic information of a patient can be received, such as using a signal receiver circuit. The physiologic information of the patient can include at least one of respiration information (e.g., respiratory rate, tidal volume, RSBI, etc.), cardiac electrical information (e.g., heart rate, impedance, etc.), impedance information, cardiac acceleration information (e.g., heart sounds, etc.), mechanical acceleration information (e.g., activity information, heart sounds, etc.), mechanical position information (e.g., patient posture, sleep incline, etc.), or other physiologic information of the patient.

At step 302, the health index can be determined, such as determined as a function of the received physiologic information, as otherwise discussed herein, such as using an assessment circuit. In an example, the function can include features, which can include functions of the physiologic information received by the signal receiver circuit. For example, a feature corresponding to a breathing rate could be calculated with a high feature value corresponding to a healthy breathing rate and low feature value corresponding to an unhealthy breathing rate. A high breathing rate (e.g., 30 breaths per minute) can be assigned a value of 100 and a low breathing rate (e.g., 12 breaths per minute) can be assigned a value of 0. The function mapping between the high breathing rate and low breathing rate can be assigned one or more of linearly, logarithmically, etc. The health index can be determined as a function of different features or combinations of physiologic information, such as one or more of a weighted combination (e.g., average, product, summation, etc.) of two or more features (e.g., each feature having a corresponding weight). The combination can include a linear combination or one or more non-linear or other combinations.

At step 303, chemical information of a patient can be received, such as using a signal receiver circuit. The chemical information can include information about chemicals within or other properties of a patient's blood and/or interstitial space, such as is otherwise described herein.

At step 304, an alert state can be determined (e.g., an in-alert state, an out-of-alert state, a priority alert state, etc.) using the determined health index and the received chemical information, such as using the assessment circuit. In an example, the alert state may be determined using the health index, such as the HeartLogic™ index, and one or more features of the received chemical information. In an example, the health index may include one or more features based on the received chemical information, and an alert state determined based on the health index may therefore be an alert state based on the health index and the received chemical information. In an example, the health index may not include any features based on the received chemical information, and an alert state based solely on the health index may therefore not be an alert state based on the health index and the received chemical information. In this case, an initial alert based on the health index may be adjusted or augmented using the received chemical information to arrive at an alert based on the chemical information.

An indication of the determined alert state may be provided to the patient, the patient's doctor, etc. In an example, an alert can be generated and provided of a transition or adjustment from the out-of-alert state to the in-alert state. The alert may be provided at a specified urgency based on a determined priority of the alert state (e.g., audible, visual, or haptic alarming, urgent notifications, etc.). If an out-of-alert state is determined, the alert state may be rechecked at an interval, such as a set interval (e.g., 1 minute, 5 minutes, 30 minutes, 1 hour, 12 hours, or 1 day). If an in-alert state is determined, the system may remain in an in-alert state until one or more of the alert is reset or the system determines that an out-of-alert state is appropriate (e.g., the health index value falls below an out-of-alert threshold). During an in-alert state, the power consumption of a device may be increased, such as may be due to one or more of a power required to generate and/or transmit one or more alerts, an increased monitoring interval, an increased processor load, etc.

At step 305, the alert based on chemical information may optionally include determining a first alert state and a second alert state. For example, the first alert state may be determined by comparing the health index value determined in step 302 to a health index alert threshold. If the health index is a specified side of the threshold (e.g., higher than the threshold, lower than the threshold), an in-alert state may be determined. Following the determination of the first alert state, the system may generate a second alert state based on the determined first alert state and one or more features of the received chemical information. For example, the system may determine the second alert state by adjusting the determined first alert state (e.g., adjusting an in-alert state to an out-of-alert state, adjusting an in-alert state to a priority alert state, maintaining the first alert state, etc.), by augmenting the determined first alert state (e.g., appending one or more pieces of additional information to the alert state), etc. In an example, the system may determine the second alert state based on one or more of the determined first alert state, one or more features of the received chemical information, the health index (e.g., the health index value), one or more other pieces of received physiologic information, one or more features used in the health index, a historical value of one or more features, etc.

At step 306, determining the alert based on chemical information may optionally include determining first and second chemical parameters. The chemical parameters may include chemical values determined based on the received chemical information. The first chemical parameter may include the chemical information in a raw form (e.g., a sensor output, such as an electrical signal, a chemical value in base units (e.g., concentration, etc.), etc.), or the first chemical parameter may be determined by processing the received chemical information (e.g., converting a sensor output to a chemical value, adjusting the chemical value based on one or more factors (e.g., adjusting to account for a temperature, pressure, other chemical concentration, etc.), etc.), etc. In an example, the second chemical parameter can be determined, such as may include determining the second chemical parameter similarly to the first chemical parameter. The alert state determined at step 304 may be determined at least in part using one or more of the first chemical parameter, the second chemical parameter, or an additional chemical parameter. In an example, the first chemical parameter may represent a potassium level and the second chemical parameter may represent a creatinine level.

At step 307, determining the first and second chemical parameters may optionally include determining the second alert state based on at least one of the determined first and second chemical parameters. For example, the first chemical parameter may be compared to a first threshold corresponding to one or more of a relative high value (e.g., a value at and/or above which the chemical parameter is determined to be outside of a normal level) or a relative low value (e.g., a value at and/or below which the chemical parameter is determined to be outside of a normal level). The first chemical parameter may also be compared to a second threshold corresponding to one or more of a relative high value or a relative low value. Relative values can include a percentage change from a patient baseline (e.g., a value that is 30 percent above a patient baseline may represent a relative high value, a value that is 30 percent below a patient baseline may represent a relative low value, etc.), a deviation from a short and/or long-term average greater than a threshold (e.g. a deviation above a long-term average value greater than a specified threshold may represent a relative high value, etc.), a value above or below one or more patient-specific or population thresholds (e.g., a high threshold may be determined for a specific patient or for a specified population), or combinations or permutations thereof. The result of the one or more comparisons may be used to determine the alert state based on chemical information at step 304, such as determining the second alert state at step 305.

At step 308, determining the alert state may optionally include providing an output of the determined alert state. For example, the output of the alert state may be provided to a patient, a clinician, or another system. The output may be provided to a user interface for display to a user or to a control circuit to control or adjust a process or function of a medical device system. For example, the output may automatically adjust a medical device system that is providing one or more therapies (e.g., medication administration, such as those described herein, cardiac rhythm management, etc.) to the patient.

In certain examples, the techniques of any one or more of steps 305-308 can be used in various combinations or permutations. In certain examples, any one or more of steps 305-308 can apply to more than one piece of chemical information.

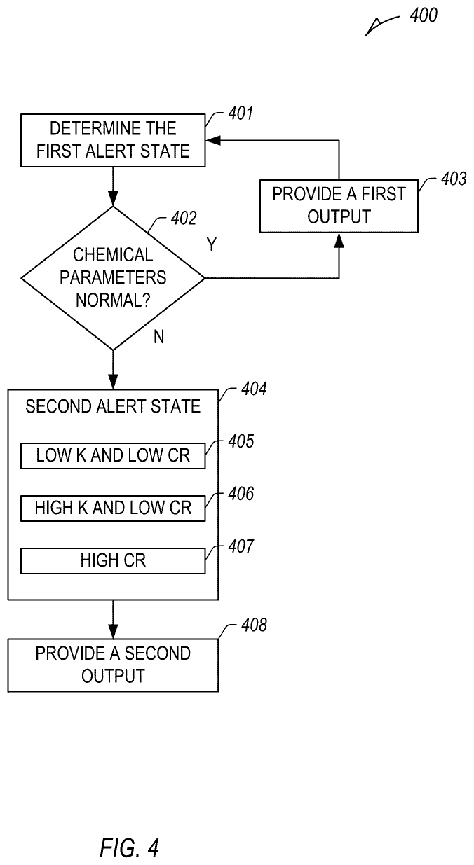

FIG. 4 illustrates an example method 400 for using chemical information to determine a second alert state, such as described herein.

At step 401, the first alert state may be determined, such as described above with respect to step 305. For example, the first alert state may be determined by comparing the health index value determined in step 302 to a health index alert threshold. If the health index is on a specified side of the threshold (e.g., higher than the threshold, lower than the threshold), an in-alert state may be determined.

At step 402, one or more chemical parameters, such as the chemical parameters determined in step 306, may be compared to one or more thresholds to determine if the chemical parameters are within a normal range. For example, a chemical parameter may be compared to a first threshold corresponding to one or more of a relative high value (e.g., a value at and/or above which the chemical parameter is determined to be outside of a normal level) or a relative low value (e.g., a value at and/or below which the chemical parameter is determined to be outside of a normal level). The first chemical parameter may also be compared to a second threshold corresponding to one or more of a relative high value or a relative low value. In an example, the first threshold may represent a relative low value and the second threshold may represent a relative high value. The chemical parameter may be determined to be normal if it is above the first threshold and below the second threshold.

At step 403, a first output may be provided if the one or more chemical parameters are determined to be normal. For example, the first output may include an output of the first alert state before chemical information was considered. In an example, the first output may include an indication that the chemical information was considered and is in the normal range. The first output may be provided to a user interface for display to a user or to a control circuit to control or adjust a process or function of a medical device system.

At step 404, a second alert state may be determined, such as described above with respect to step 305, if the chemical information is not normal. The second alert state may include one of a plurality of guideline-directed medical therapy (GDMT) alert states associated with a therapy adjustment. For example, the second alert state may include an indication to a system or clinician of a class of drug to administer or provide to the patient.

At step 405, the second alert state may provide a control signal to provide or increase a potassium sparing diuretic when the determined first chemical parameter indicates that a potassium level is below a relative low potassium value (e.g., below a potassium threshold) and/or the determined second chemical parameter indicates that a creatinine value is below a relative low creatinine value (e.g., below a creatinine threshold).

At step 406, the second alert state may provide a control signal to provide or increase a thiazide diuretic when the determined first chemical parameter indicates that a potassium level is above a relative high potassium value (e.g., above a potassium threshold) and/or the determined second chemical parameter indicates that a creatinine value is below a relative low creatinine value (e.g., below a creatinine threshold).

At step 407, the second alert state may provide a control signal to provide or increase a vasodilator when the determined second chemical parameter indicates that a creatinine value is above a relative high creatinine value (e.g., above a creatinine threshold).

At step 408, a second output corresponding to the second alert state may be provided. The second output may be provided as an alternative to, or in addition to, the first output. The second output may be provided to a user interface for display to a user or to a control circuit to control or adjust a process or function of a medical device system.

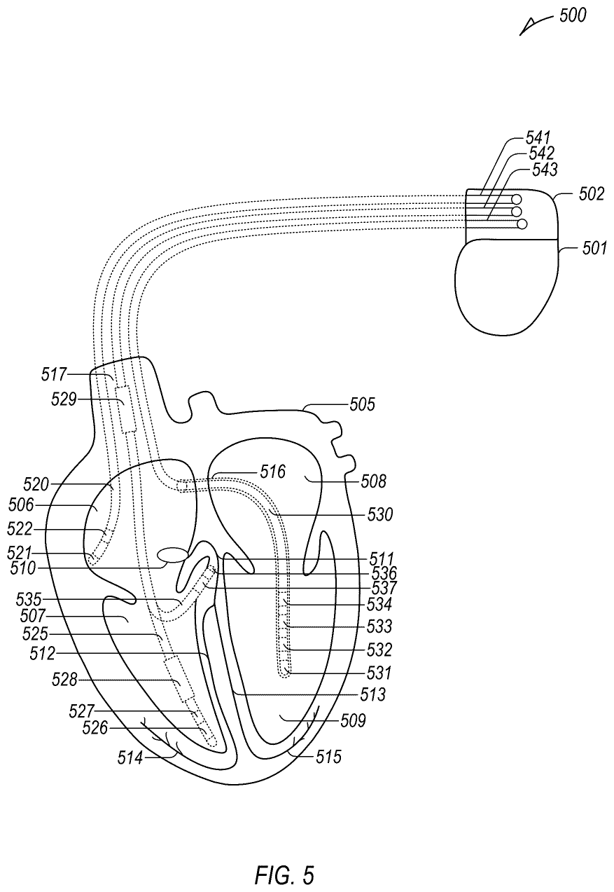

FIG. 5 illustrates an implantable medical device (IMD) 500 electrically coupled to a heart 505, such as through one or more leads coupled to the IMD 500 through one or more lead ports, including first, second, or third lead ports 541, 542, 543 in a header 502 of the IMD 500. In an example, the IMD 500 can include an antenna, such as in the header 502, configured to enable communication with an external system and one or more electronic circuits (e.g., an assessment circuit, etc.) in a hermetically sealed housing (CAN) 501. The IMD 500 illustrates an example medical device (or a medical device system) as described herein.

The IMD 500 may include an implantable medical device (IMD), such as an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 505, such as in one or more of the atria or ventricles. Separate from, or in addition to, the one or more electrodes or other sensors of the leads, the IMD 500 can include one or more electrodes or other sensors (e.g., a pressure sensor, an accelerometer, a gyroscope, a microphone, etc.) powered by a power source in the IMD 500. The one or more electrodes or other sensors of the leads, the IMD 500, or a combination thereof, can be configured detect physiologic information from, or provide one or more therapies or stimulation to, the patient.

The IMD 500 can include one or more electronic circuits configured to sense one or more physiologic signals, such as an electrogram or a signal representing mechanical function of the heart 505. In certain examples, the CAN 501 may function as an electrode such as for sensing or pulse delivery. For example, an electrode from one or more of the leads may be used together with the CAN 501 such as for unipolar sensing of an electrogram or for delivering one or more pacing pulses. A defibrillation electrode (e.g., the first defibrillation coil electrode 528, the second defibrillation coil electrode 529, etc.) may be used together with the CAN 501 to deliver one or more cardioversion/defibrillation pulses.

In an example, the IMD 500 can sense impedance such as between electrodes located on one or more of the leads or the CAN 501. The IMD 500 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the IMD 500 can be configured to inject current between an electrode on one or more of the first, second, third, or fourth leads 520, 525, 530, 535 and the CAN 501, and to sense the resultant voltage between the same or different electrodes and the CAN 501.

The example lead configurations in FIG. 5 include first, second, and third leads 520, 525, 530 in traditional lead placements in the right atrium (RA) 506, right ventricle (RV) 507, and in a coronary vein 516 (e.g., the coronary sinus) over the left atrium (LA) 508 and left ventricle (LV) 509, respectively, and a fourth lead 535 positioned in the RV 507 near the His bundle 511, between the AV node 510 and the right and left bundle branches 512, 513 and Purkinje fibers 514, 515. Each lead can be configured to position one or more electrodes or other sensors at various locations in or near the heart 505 to detect physiologic information or provide one or more therapies or stimulation.

The first lead 520, positioned in the RA 506, includes a first tip electrode 521 located at or near the distal end of the first lead 520 and a first ring electrode 522 located near the first tip electrode 521. The second lead 525 (dashed), positioned in the RV 507, includes a second tip electrode 526 located at or near the distal end of the second lead 525 and a second ring electrode 527 located near the second tip electrode 526. The third lead 530, positioned in the coronary vein 516 over the LV 509, includes a third tip electrode 531 located at or near the distal end of the third lead 530, a third ring electrode 532 located near the third tip electrode 531, and two additional electrodes 533, 534. The fourth lead 535, positioned in the RV 507 near the His bundle 511, includes a fourth tip electrode 536 located at or near the distal end of the fourth lead 535 and a fourth ring electrode 537 located near the fourth tip electrode 536. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 525 includes a first defibrillation coil electrode 528 located near the distal end of the second lead 525 in the RV 507 and a second defibrillation coil electrode 529 located a distance from the distal end of the second lead 525, such as for placement in or near the superior vena cava (SVC) 517.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 511.

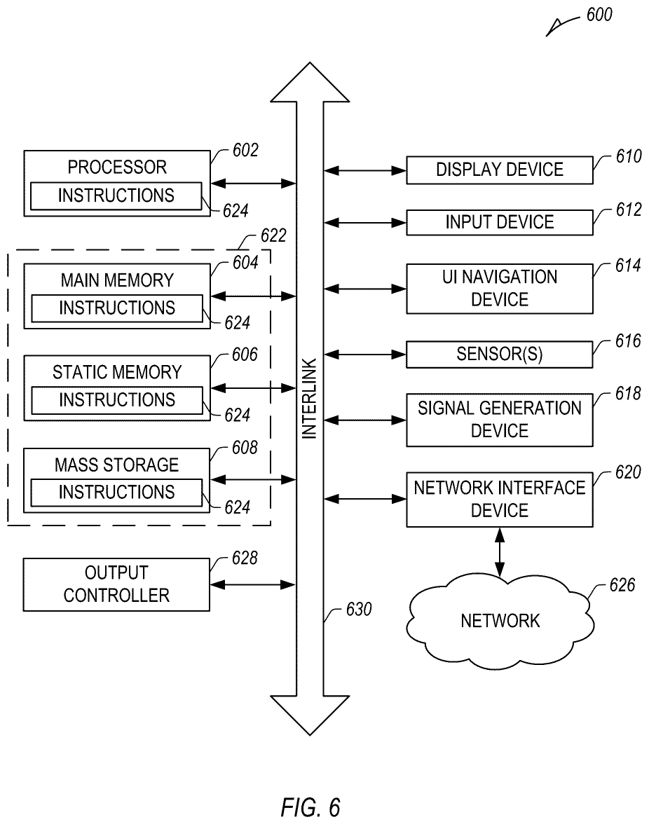

FIG. 6 illustrates a block diagram of an example machine 600 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 600. Circuitry (e.g., processing circuitry, an assessment circuit, etc.) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 600 (e.g., computer system) may include a hardware processor 602 (e.g., a central processing unit (CPU), a graphics processing unit (GPU), a hardware processor core, or any combination thereof), a main memory 604, a static memory 606 (e.g., memory or storage for firmware, microcode, a basic-input-output (BIOS), unified extensible firmware interface (UEFI), etc.), and mass storage 608 (e.g., hard drive, tape drive, flash storage, or other block devices) some or all of which may communicate with each other via an interlink 630 (e.g., bus). The machine 600 may further include a display unit 610, an input device 612 (e.g., a keyboard), and a user interface (UI) navigation device 614 (e.g., a mouse). In an example, the display unit 610, input device 612, and UI navigation device 614 may be a touch screen display. The machine 600 may additionally include a signal generation device 618 (e.g., a speaker), a network interface device 620, and one or more sensors 616, such as a global positioning system (GPS) sensor, compass, accelerometer, or one or more other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 602, the main memory 604, the static memory 606, or the mass storage 608 may be, or include, a machine-readable medium 622 on which is stored one or more sets of data structures or instructions 624 (e.g., software) embodying or utilized by any one or more of the techniques or functions described herein. The instructions 624 may also reside, completely or at least partially, within any of registers of the hardware processor 602, the main memory 604, the static memory 606, or the mass storage 608 during execution thereof by the machine 600. In an example, one or any combination of the hardware processor 602, the main memory 604, the static memory 606, or the mass storage 608 may constitute the machine-readable medium 622. While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a signal receiver circuit configured to receive physiologic information of a patient; and
an assessment circuit configured to determine a health index for the patient as a function of the received physiologic information,
wherein the signal receiver circuit is configured to receive chemical information of the patient, and
wherein the assessment circuit is configured to determine an alert state of the patient using the determined health index and the received chemical information.

2. The medical device system of claim 1, wherein the health index includes a composite health index, wherein the assessment circuit is configured to determine the composite health index for the patient as a function of at least two features of the received physiologic information.

3. The medical device system of claim 2, wherein, to determine the alert state of the patient, the assessment circuit is configured to:
determine a first alert state of the patient using a value of the determined health index and a health index alert threshold; and
determine a second alert state based on the determined first alert state and the received chemical information of the patient.

4. The medical device system of claim 1, wherein, to determine the alert state of the patient, the assessment circuit is configured to:
determine a first alert state of the patient using a value of the determined health index and a health index alert threshold; and
determine a second alert state based on the determined first alert state and the received chemical information of the patient.

5. The medical device system of claim 4, wherein the second alert state is one of a plurality of guideline-directed medical therapy (GDMT) alert states associated with a therapy adjustment,
wherein the assessment circuit is configured to provide an output of the determined alert state to a user interface for display to a user or to a control circuit to control or adjust a process or function of the medical device system.

6. The medical device system of claim 5, wherein the assessment circuit is configured to determine first and second chemical parameters using the received chemical information,
wherein the received chemical information comprises at least one of potassium information or creatinine information of the patient,
wherein the first and second chemical parameters comprise indications of relative high or low values of the received chemical information with respect to one or more thresholds,
wherein to determine the first alert state, the assessment circuit is configured to determine that the value of the determined health index is above the health index alert threshold, and
wherein the assessment circuit is configured to determine the second alert state based on the determined first alert state and at least one of the determined first and second chemical parameters.

7. The medical device system of claim 6, wherein to determine the second alert state includes to determine that the potassium information of the patient is below a potassium threshold and the creatinine information of the patient is below a creatinine threshold, and
wherein the assessment circuit is configured to provide a control signal to provide or increase a potassium sparing diuretic in response to determining the second alert state.

8. The medical device system of claim 6, wherein to determine the second alert state includes to determine that the potassium information of the patient is above a potassium threshold and the creatinine information of the patient is below a creatinine threshold, and
wherein the assessment circuit is configured to provide a control signal to provide or increase a thiazide diuretic in response to determining the second alert state.

9. The medical device system of claim 6, wherein to determine the second alert state includes to determine that the creatinine information of the patient is above a creatinine threshold, and
wherein the assessment circuit is configured to provide a control signal to provide or increase a vasodilator in response to determining the second alert state.

10. The medical device system of claim 4, wherein the health index includes a heart failure index and the health index alert threshold includes a heart failure alert threshold.

11. A method, comprising:
receiving, using a signal receiver circuit, physiologic information of a patient,
determining, using an assessment circuit, a health index for the patient as a function of the received physiologic information,
receiving, using the signal receiver circuit, chemical information of a patient, and
determining, using the assessment circuit, an alert state of the patient using the determined health index and the received chemical information.

12. The method of claim 11, wherein the health index comprises a composite health index, and wherein determining the health index includes determining the composite health index for the patient as a function of at least two features of the received physiologic information.

13. The method of claim 12, wherein determining the alert state of the patient includes:
determining a first alert state of the patient using a value of the determined health index and a health index alert threshold, and
determining a second alert state based on the determined first alert state and the received chemical information of the patient.

14. The method of claim 11, wherein determining the alert state of the patient includes:
determining a first alert state of the patient using a value of the determined health index and a health index alert threshold, and
determining a second alert state based on the determined first alert state and the received chemical information of the patient.

15. The method of claim 14, comprising:
providing, using the assessment circuit, an output of the determined alert state to a user interface for display to a user or to a control circuit to control or adjust a process or function of a medical device system,
wherein the second alert state is one of a plurality of guideline-directed medical therapy (GDMT) alert states associated with a therapy adjustment.

16. The method of claim 15, comprising determining, using the assessment circuit, first and second chemical parameters using the received chemical information,
wherein the received chemical information comprises at least one of potassium information or creatinine information of the patient,
wherein the first and second chemical parameters comprise indications of relative high or low values of the received chemical information with respect to one or more thresholds,
wherein determining the first alert state includes determining that the value of the determined health index is above the health index alert threshold, and
wherein determining the second alert state includes determining the second alert state based on the determined first alert state and at least one of the determined first and second chemical parameters.

17. The method of claim 16, wherein determining the second alert state includes determining that the potassium information of the patient is below a potassium threshold and the creatinine information of the patient is below a creatinine threshold, and
providing, in response to determining the second alert state, a control signal to provide or increase a potassium sparing diuretic.

18. The method of claim 16, wherein determining the second alert state includes determining that the potassium information of the patient is above a potassium threshold and the creatinine information of the patient is below a creatinine threshold, and
providing, in response to determining the second alert state, a control signal to provide or increase a thiazide diuretic.

19. The method of claim 16, wherein determining the second alert state includes determining that the creatinine information of the patient is above a creatinine threshold, and
providing, in response to determining the second alert state, a control signal to provide or increase a vasodilator.

20. The method of claim 14, wherein the health index includes a heart failure index and the health index alert threshold includes a heart failure alert threshold.
